(19) United States
(12) Patent Application Publication
(10) Pub. No.: US 2015/0374297 A1
(43) Pub. Date: Dec. 31, 2015

(54) MODULAR INSTRUMENTED FLOOR COVERING

(71) Applicant: Clinical Image Retrieval Systems, Inc., Sparta, NJ (US)

(72) Inventors: Douglas D. Haas, Sparta, NJ (US); Igor Ofenbakh, Morristown, NJ (US)

(21) Appl. No.: 14/318,035

(22) Filed: Jun. 27, 2014

(51) Int. Cl. A61B 5/00; A61B 5/11; G01V 9/00

(52) U.S. Cl. CPC A61B 5/6887; G01V 9/00; A61B 5/112; A61B 5/1113; A61B 5/0022; A61B 2562/0247; A61B 2560/0443

(57) ABSTRACT

A modular instrumented floor covering assembly is used in connection with a subject walking across the assembly. The floor covering assembly comprises a plurality of sensor panels having interlocking edges. The sensor panels are adapted for interlocking the adjacent panels together along the edges. Each sensor panel has a pressure sensor matrix responsive to a weight of the subject for generating data relating to movement of the subject. The sensor panels are adapted for selective and releasable assembly in patterns. Communicating means is provided for wirelessly communicating data between the sensor panels and from the sensor panels to a computer for analysis. Power means is provided for supplying power to the sensor panels and between adjacent sensor panels.

### INCORPORATION BY REFERENCE

U.S. Pat. No. 5,952,585, issued Sep. 14, 1999, entitled “Portable Pressure Sensing Apparatus For Measuring Dynamic Gait Analysis And Method Of Manufacture,” is incorporated in its entirety for the teachings therein.

### TECHNICAL FIELD

The presently disclosed technologies are directed to an apparatus and method for a pressure sensitive instrumented floor, and in particular, a plurality of modular, interlocking, instrumented panels that fit together selectively, over which subjects walk for data collection.

### BACKGROUND

The collection of data for subjects walking upon a floor is accomplished by laying out a pressure sensitive instrument panel. The subject walks along the panel, and data is communicated to a computer by hard wiring. This is routinely used for analyzing the gait of humans or animals. The apparatus is an over ground system using a long pressure sensor matrix laid under a carpeted walkway, which in recent years has proven to be highly accurate and easy to use in both research and clinical practice.

Such a pressure sensitive instrument panel can be used for medical and veterinary diagnosis of walking problems. It can also be used for security, to determine in real time where a subject is and in what direction the subject is moving within the space.

An exemplary pressure sensor matrix is found in U.S. Pat. No. 5,952,585, the disclosure of which is incorporated herein by reference. This patent is the basis of a product entitled, “GAITRITE®,” which a 2 foot wide portable walkway system with a maximum length of 26 feet. The Gaitrite apparatus is the Gold Standard in the evaluation of Pressure based Temporal/Spatial gait analysis worldwide. The Gaitrite apparatus nevertheless has limitations, including width and length restrictions. Furthermore, the system had to be directly connected to a computer via cable. This limits the ability to walk in other than a straight line or a confined U turn. Over the years many systems have attempted and failed to provide more open walking surface or easy connectivity. All these systems to date have been too restrictive in ease of installation and in flexibility of layout options. One problem has been laying down custom pathways along which the subject can walk. The pathway selections are very limited, and cannot be changed. Another problem has been wiring the pressure sensor matrix for signal and power. These systems require custom wiring under the sensors.

There is a need, therefore, for a pressure sensitive walkway for data collection which does not require any custom hard wiring.

There is a further need for a pressure sensitive walkway for data collection as described, and that has pathways that can be reconfigured selectively.

There is a yet further need for a pressure sensitive walkway for data collection as described, and that can be installed by one person with limited skills and no tools.

There is a still further need for a pressure sensitive walkway for data collection as described, and that can be monitored locally or remotely.

### SUMMARY

In one aspect, a modular instrumented floor covering assembly is used in connection with a subject walking across the assembly. The floor covering assembly comprises a plurality of sensor panels having interlocking edges. The sensor panels are adapted for interlocking the adjacent panels together along the edges. Each sensor panel has a pressure sensor matrix responsive to a weight of the subject for generating data relating to movement of the subject. The plurality of sensor panels are adapted for selective and releasable assembly in patterns. Communicating means is provided for communicating data from the sensor panels. Power means is provided for supplying power to the sensor panels and between adjacent sensor panels.

These and other aspects, objectives, features, and advantages of the disclosed technologies will become apparent from the following detailed description of illustrative embodiments thereof, which is to be read in connection with the accompanying drawings.

### BRIEF DESCRIPTION OF THE DRAWINGS

FIG. 1 is a top plan view of a modular instrumented floor covering panel constructed in accordance with the invention.

FIG. 2 is a right side view of the modular instrumented panel of FIG. 1.

FIG. 3 is an inverted rear elevational view of the modular instrumented panel of FIG. 1.

FIG. 4 is a bottom plan view of the modular instrumented panel of FIG. 1.

FIG. 5 is a left side view of the modular instrumented panel of FIG. 1.

FIG. 6 is a front elevational view of the modular instrumented panel of FIG. 1.

FIG. 7 is a cross-sectional elevational detail view of the modular instrumented panel of FIG. 1, taken along lines 7-7 of FIG. 4.

FIG. 8 is a cross-sectional elevational detail view of the modular instrumented panel of FIG. 1, taken along lines 8-8 of FIG. 1.

FIG. 9A is a cutaway top plan view of the modular instrumented panel of FIG. 1, taken along lines 9-9 of FIG. 3, and showing the layers.

FIG. 9B is a cutaway top plan view of the modular instrumented panel of FIG. 1, taken along lines 9-9 of FIG. 3, and showing the power connections in the panel.

FIG. 9C is a cutaway top plan view of the modular instrumented panel of FIG. 1, taken along lines 9-9 of FIG. 3, and showing the power connections between assembled panels.

FIG. 10 is a perspective assembly view of three of the modular instrumented panels of FIG. 1, and an edge panel, showing the assembly procedure.

FIG. 11 is an enlarged, perspective detail view of the modular instrumented panel of FIG. 1, taken at detail 11 of FIG. 10.

FIG. 12 is an enlarged, perspective detail view of the modular instrumented panel of FIG. 1, taken at detail 12 of FIG. 10.

FIG. 13 is a top plan view of an edge panel for use with the modular instrumented panel of FIG. 1.

FIG. 14 is an end view of the edge panel of FIG. 13.

FIG. 15 is an edge view of the edge panel of FIG. 13.

FIG. 16 is a top plan view of another edge panel for use with the modular instrumented panel of FIG. 1.

FIG. 17 is an end view of the edge panel of FIG. 16.

FIG. 18 is an edge view of the edge panel of FIG. 16.

FIG. 19 is a perspective view of the edge panel of FIG. 13.

FIG. 20 is a perspective view of the edge panel of FIG. 16.

FIG. 21 is a top plan view of an inert panel for use with the modular instrumented panel of FIG. 1.

FIG. 22 is an end view of the inert panel of FIG. 21.

FIG. 23 is an edge view of the inert panel of FIG. 21.

FIG. 24 is a top plan view of another inert panel for use with the modular instrumented panel of FIG. 1.

FIG. 25 is an end view of the inert panel of FIG. 24.

FIG. 26 is an edge view of the inert panel of FIG. 24.

FIG. 27 is a perspective view of the inert panel of FIG. 21.

FIG. 28 is a perspective view of the inert panel of FIG. 24.

FIG. 29 is a perspective assembly exploded view of the modular instrumented panel of FIG. 1, and two edge panels, and an inert panel showing the assembly procedure.

FIG. 30 is a perspective assembly contracted view of the assembly of FIG. 29.

FIG. 31 is a cross-sectional elevational detail view of the modular instrumented panel assembly of FIG. 10, taken along lines 31-31 of FIG. 10, and showing the interlocking strips exploded.

FIG. 32 is a cross-sectional elevational detail view of FIG. 10, taken along lines 32-32 of FIG. 10, and showing the interlocking strips assembled.

FIG. 33 is a cross-sectional elevational detail view of a modular instrumented panel assembly constructed in accordance with the invention, showing another embodiment of the interlocking strips exploded.

FIG. 34 is a cross-sectional elevational detail view of FIG. 33, showing the interlocking strips assembled.

FIG. 35 is a cross-sectional elevational detail view of the modular instrumented panel of FIG. 1, taken along lines 8-8 of FIG. 1, and showing the electrical contacts exploded.

FIG. 36 is a cross-sectional elevational detail view of the modular instrumented panel of FIG. 1, taken along lines 8-8 of FIG. 1, and showing the electrical contacts engaged.

FIG. 37 is a perspective view of the electrical contact of FIG. 35.

FIG. 38 is a side elevational detail view of the electrical contact of FIG. 35.

FIG. 39 is a top plan view of a perimeter pattern for use with the invention.

FIG. 40 is a top plan view of a a T-shaped pattern for use with the invention.

FIG. 41 is a top plan view of an area pattern for use with the invention.

FIG. 42 is a top plan view of an L-shaped pattern for use with the invention.

FIG. 43 is a top plan view of a straight pattern for use with the invention.

FIG. 44 is a top plan view of a U-shaped pattern for use with the invention.

FIG. 45 is a cross-sectional elevational detail view of yet another modular instrumented panel assembly constructed in accordance with the invention, showing yet another embodiment of the interlocking strips exploded.

FIG. 46 is a cross-sectional elevational detail view of FIG. 45, showing the interlocking strips assembled.

FIG. 47 is a cutaway top plan view of still another modular instrumented panel constructed in accordance with the invention, and showing the signal over power connections in the panel.

FIG. 48 is a cutaway top plan view of the modular instrumented panel of FIG. 47, and showing the power and signal connections between assembled panels.

FIG. 49 is a top plan view of a further modular instrumented floor covering panel constructed in accordance with the invention.

FIG. 50 is a right side view of the modular instrumented panel of FIG. 49.

FIG. 51 is a front elevational view of the modular instrumented panel of FIG. 49.

FIG. 52 is a bottom plan view of the modular instrumented panel of FIG. 49.

FIG. 53 is a right side view of the modular instrumented panel of FIG. 49.

FIG. 54 is a rear inverted elevational view of the modular instrumented panel of FIG. 49.

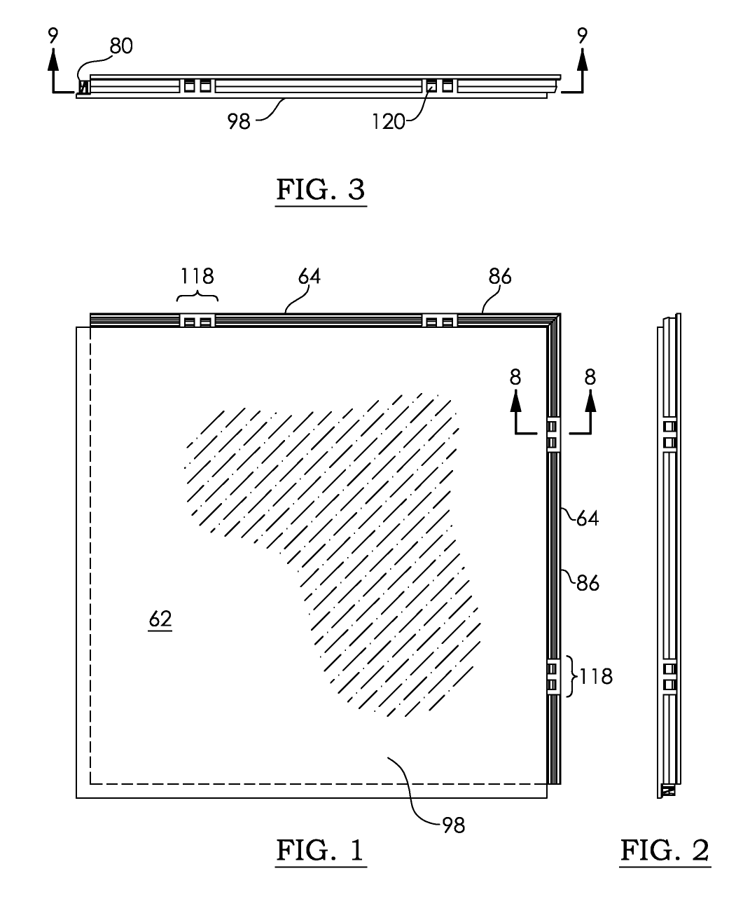

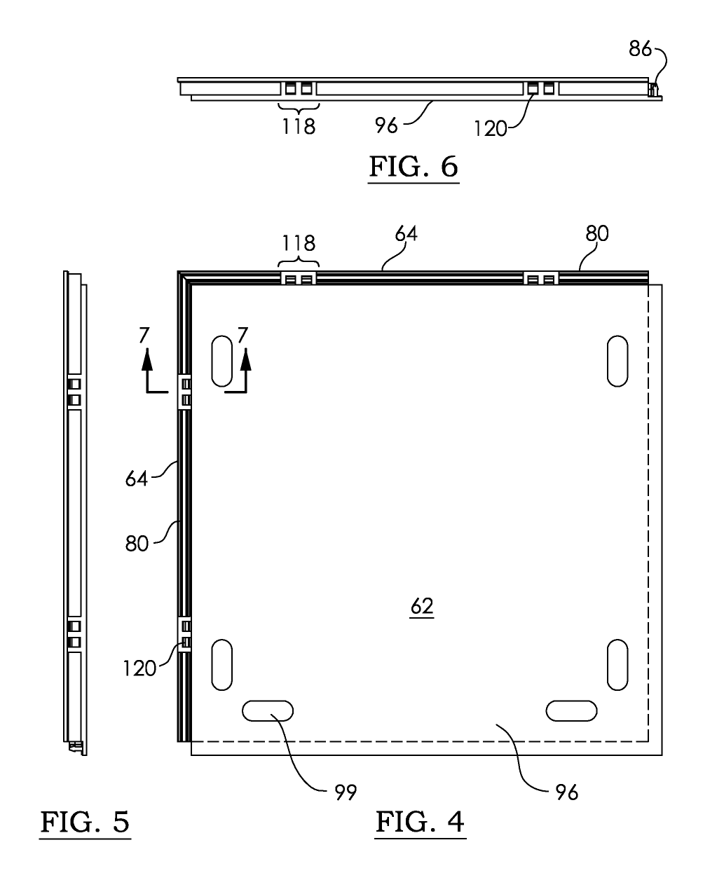

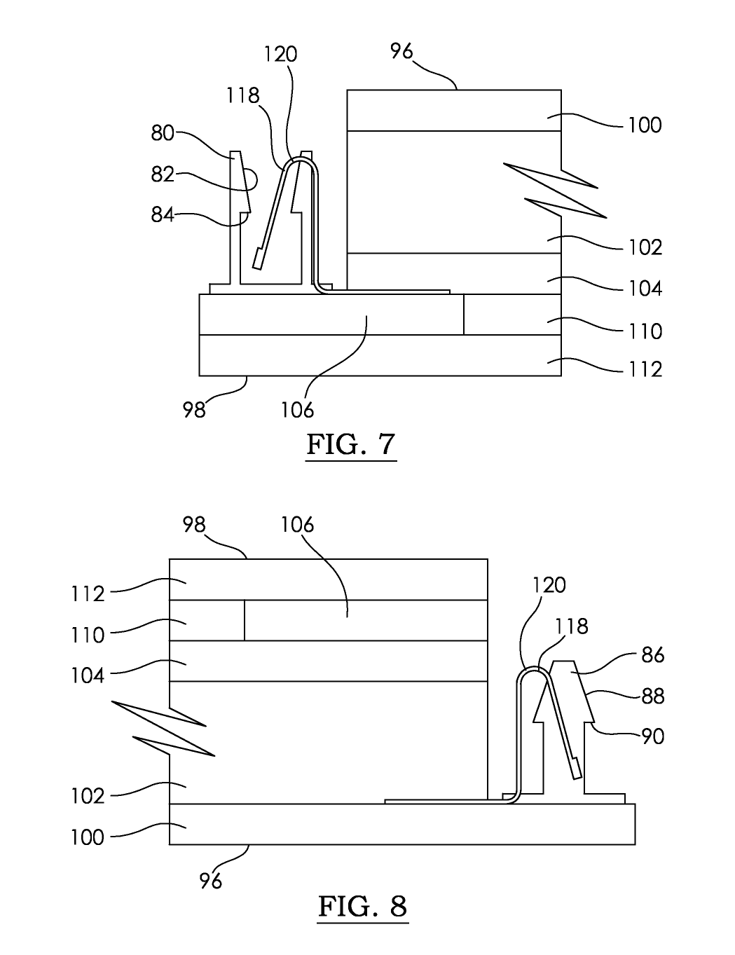

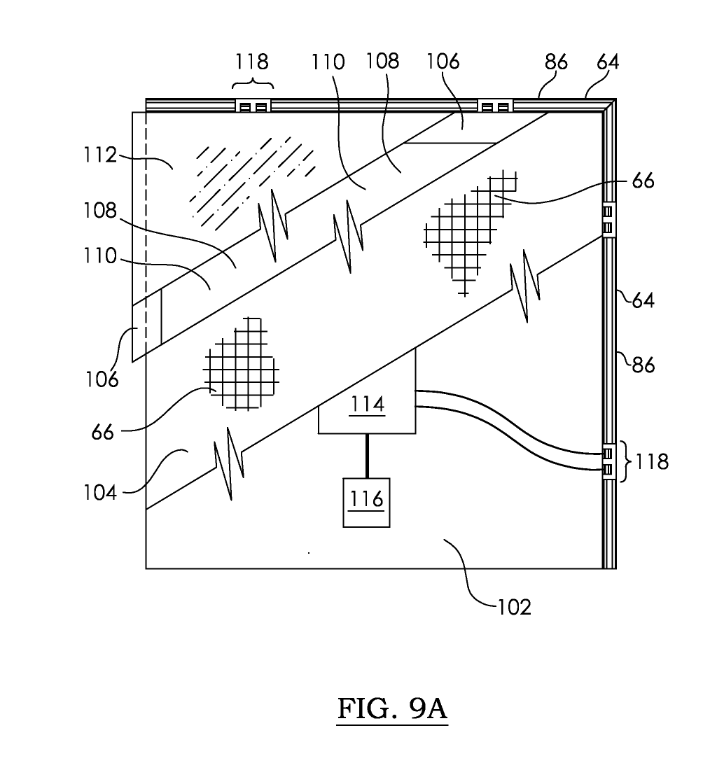

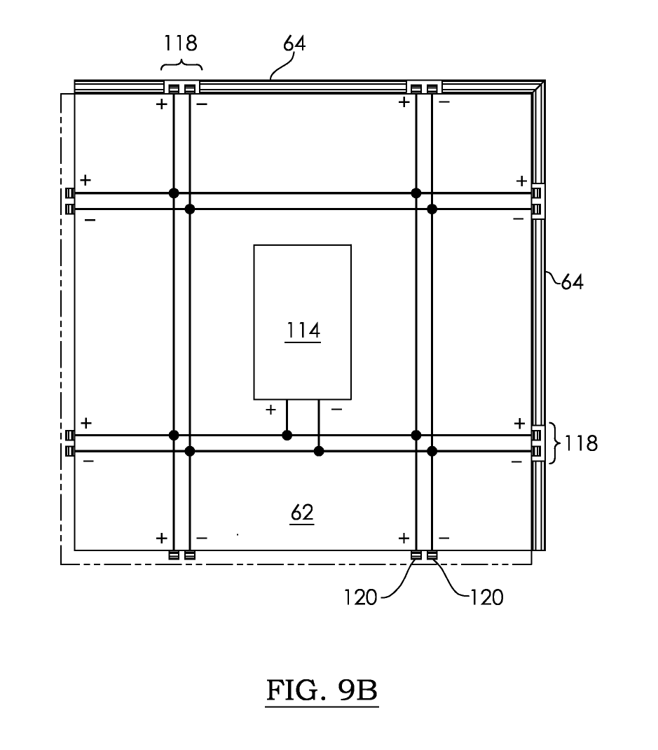

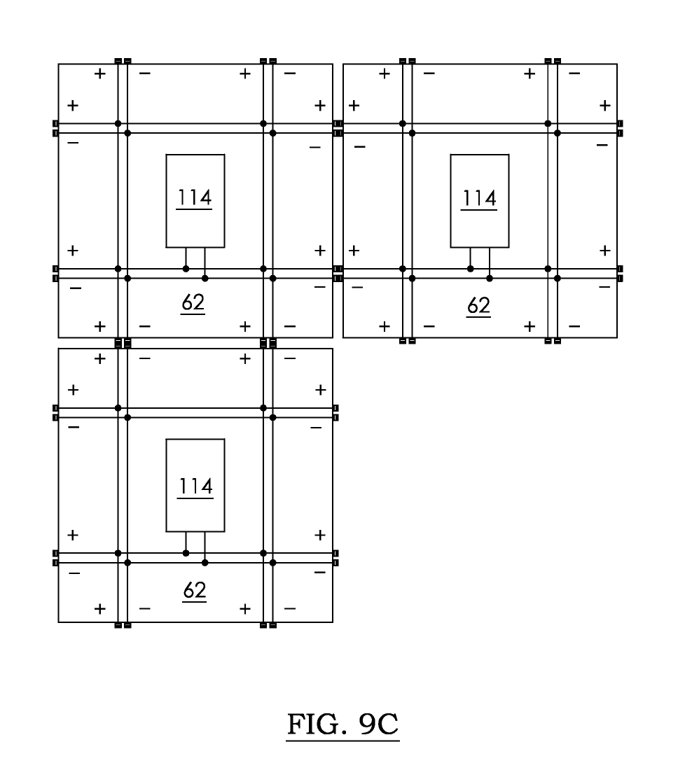

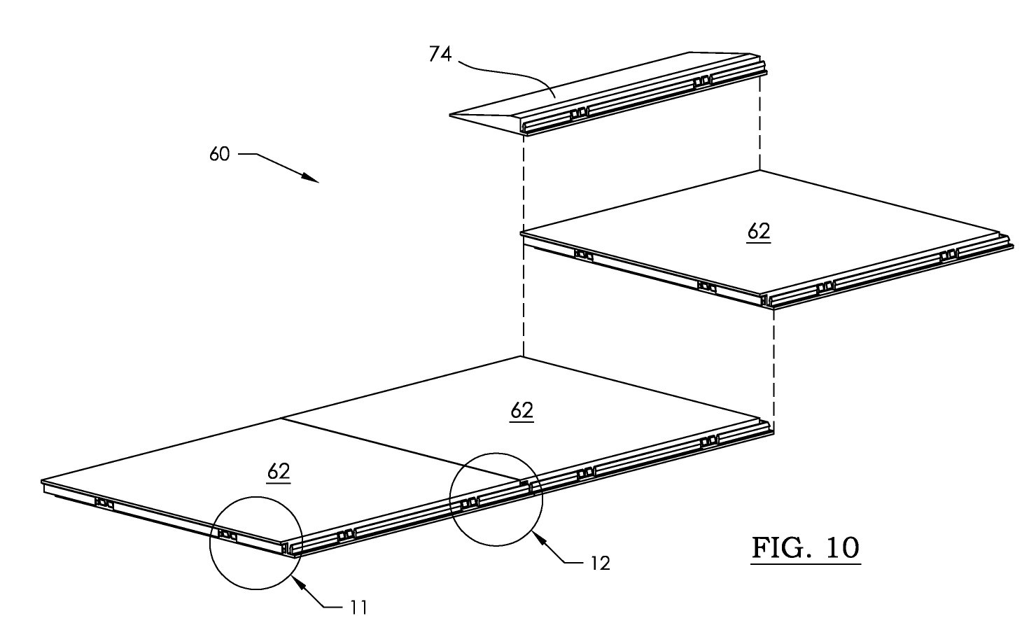

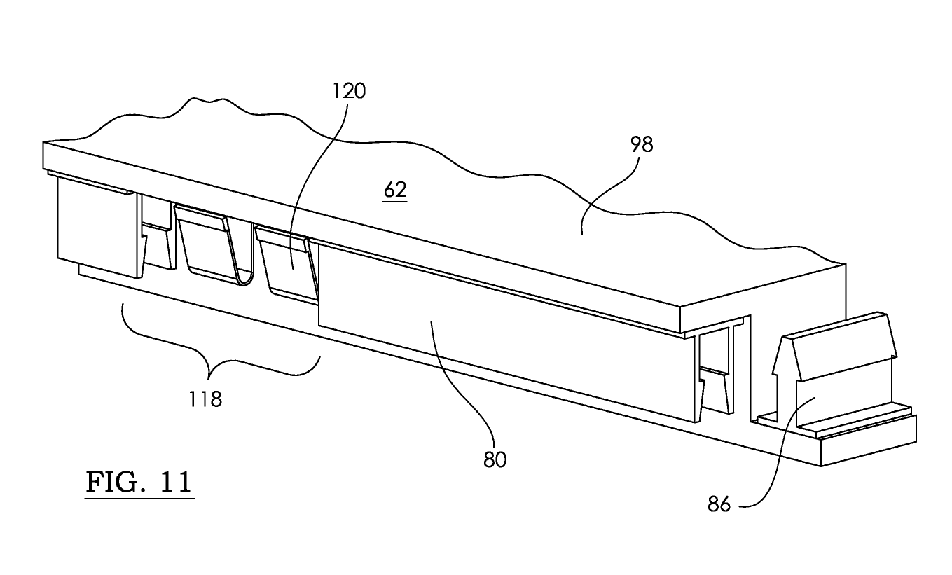

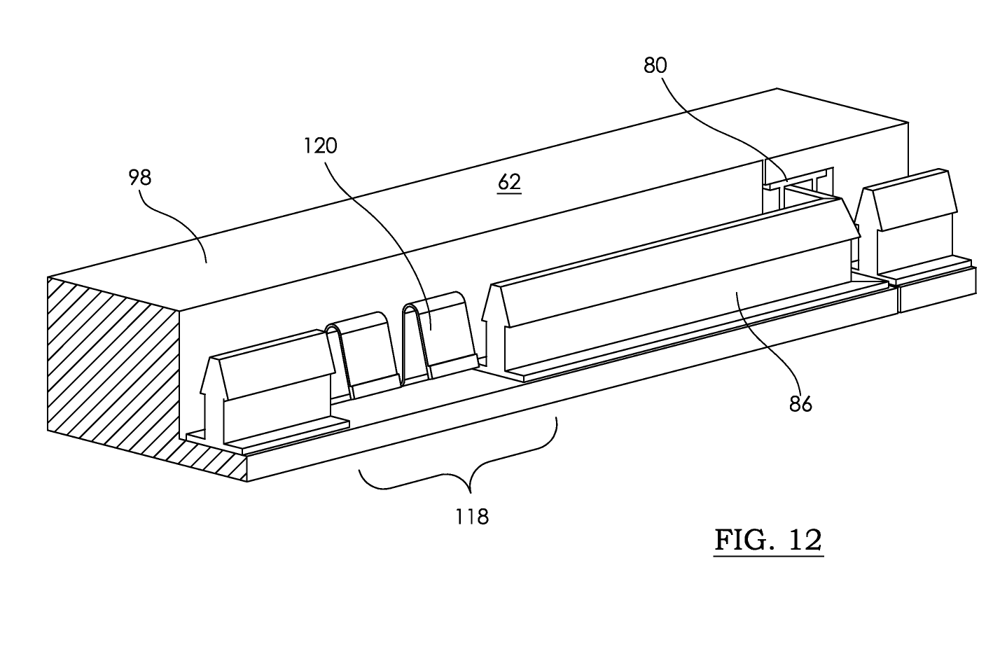

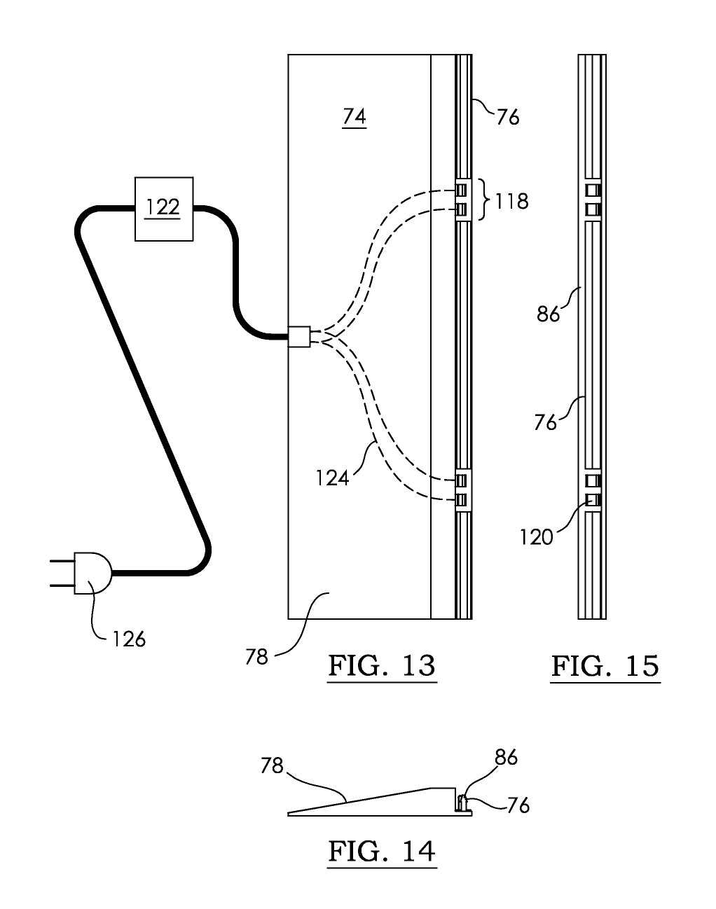

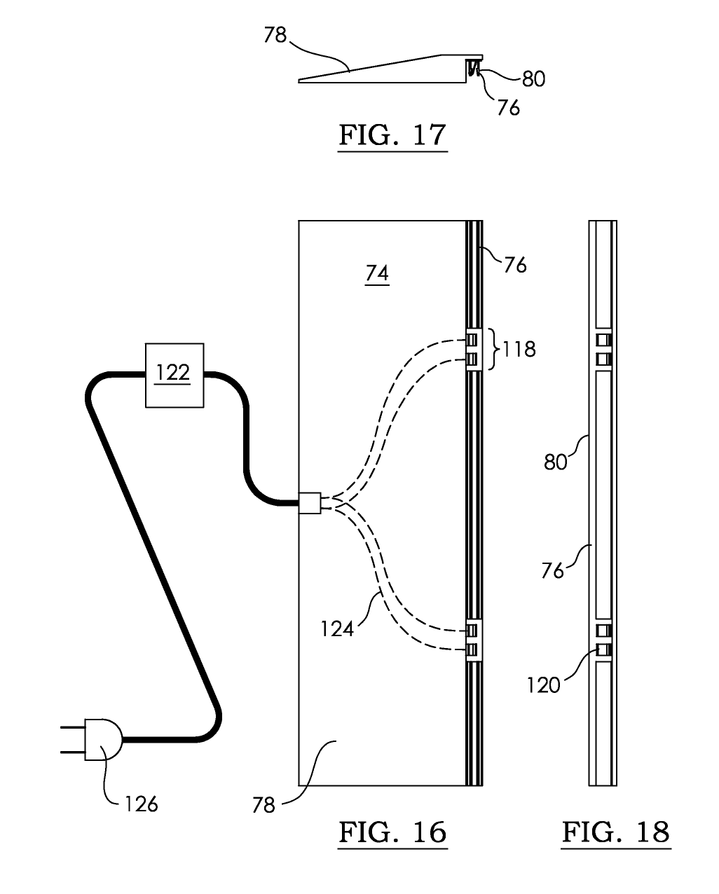

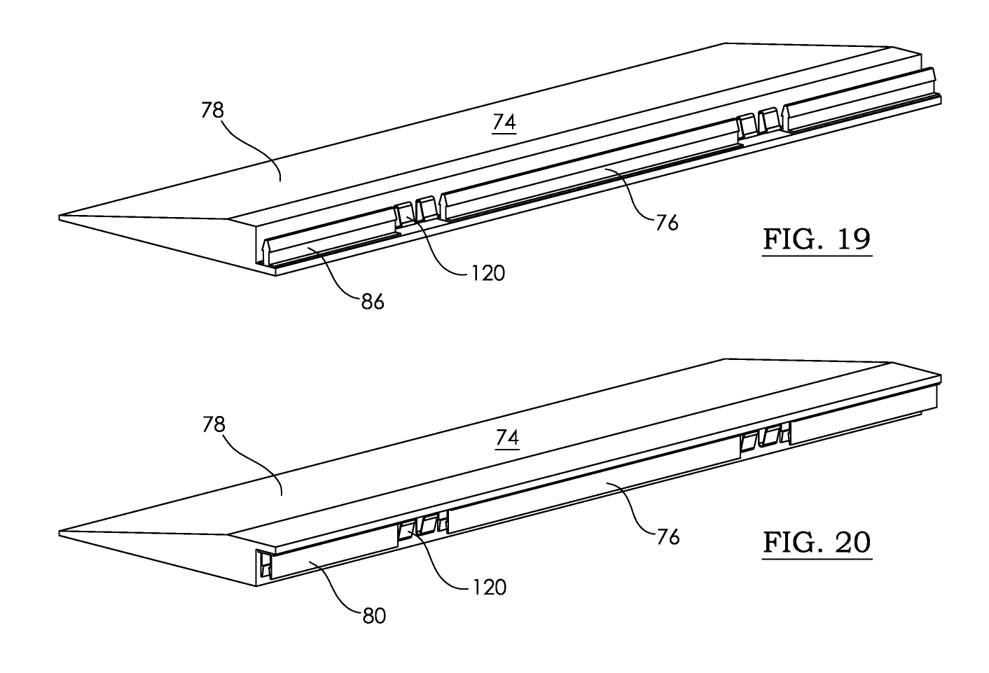

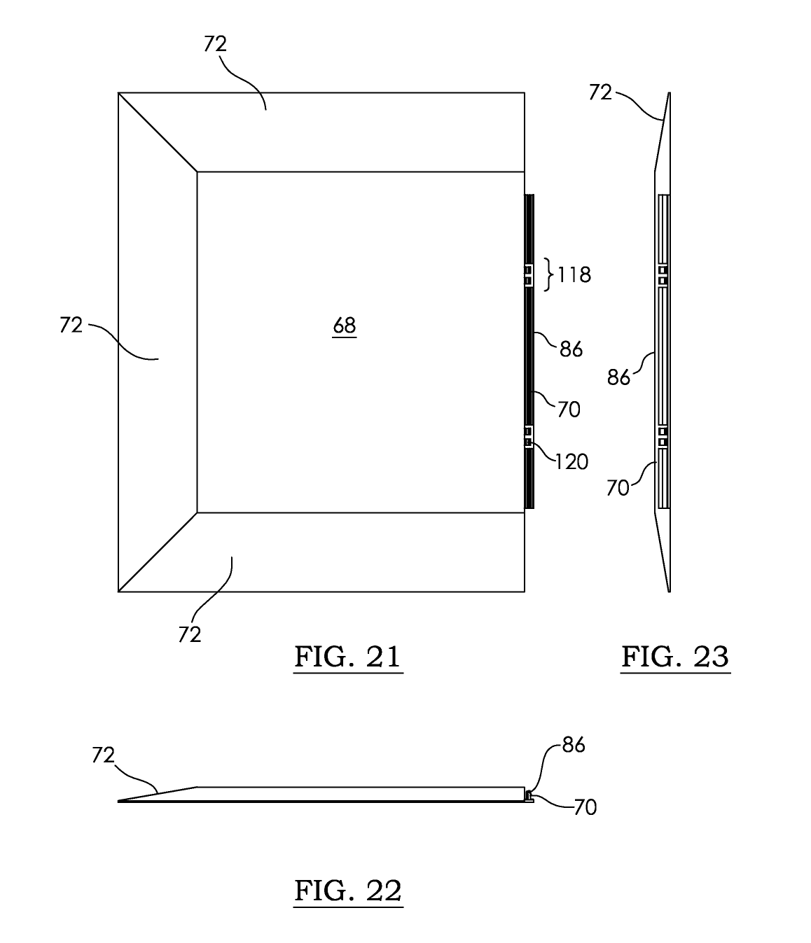

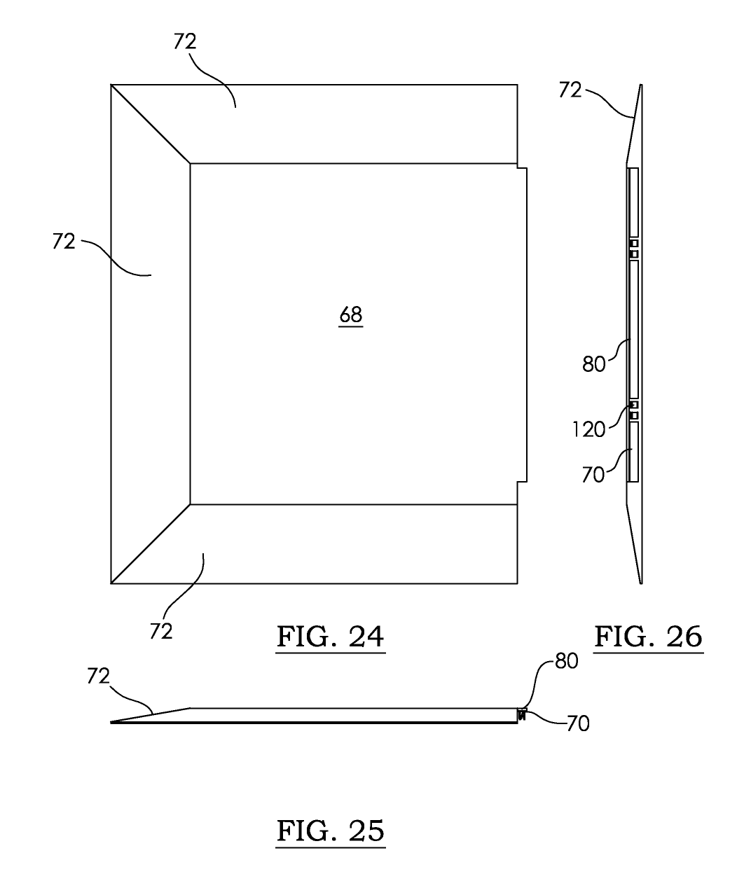

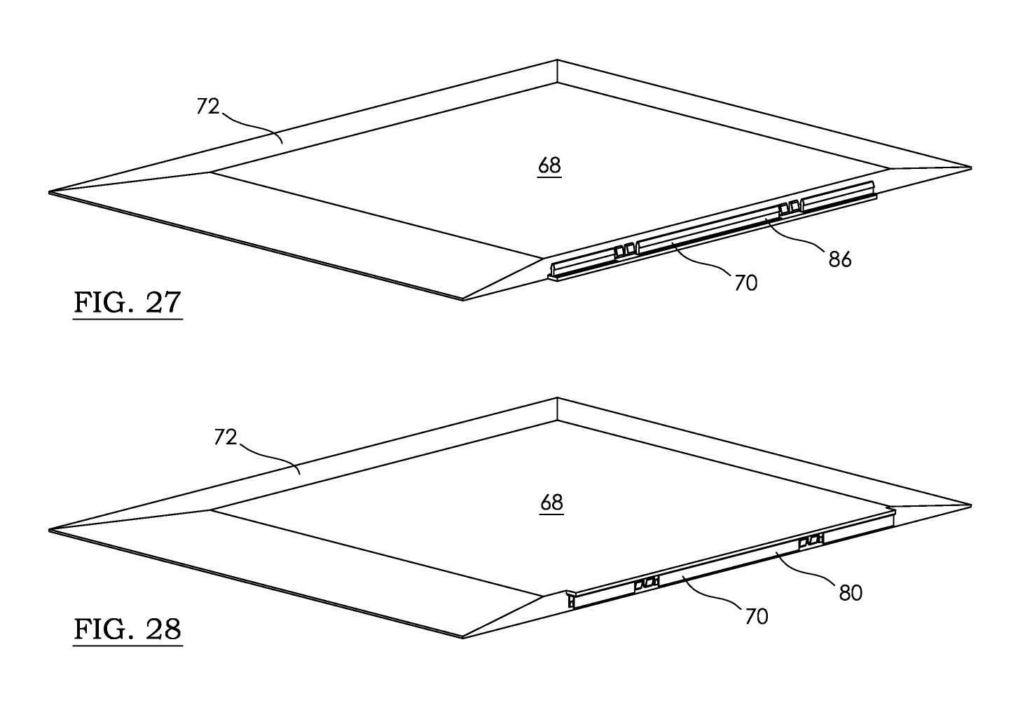

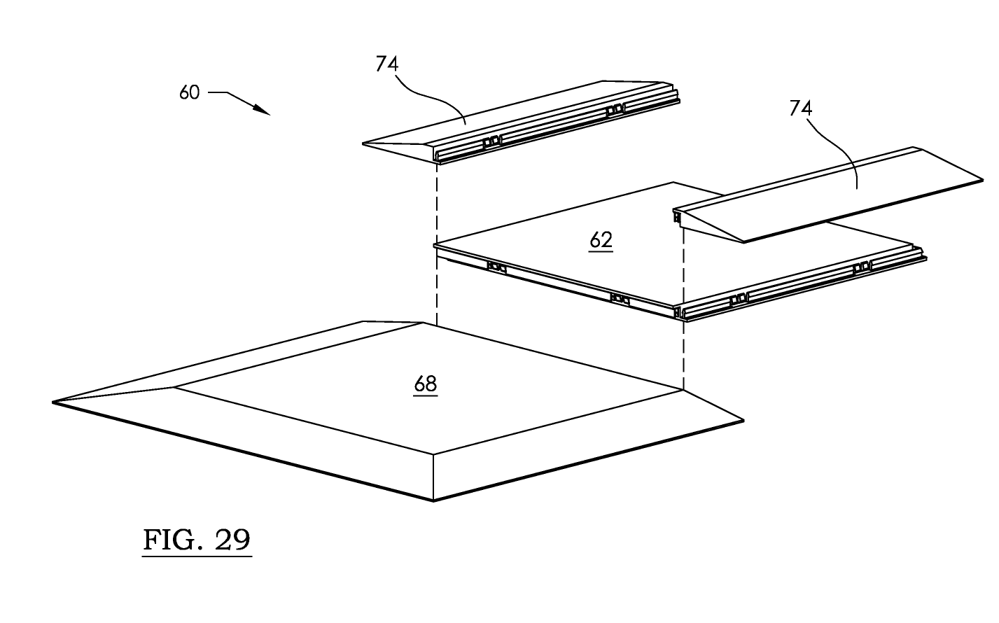

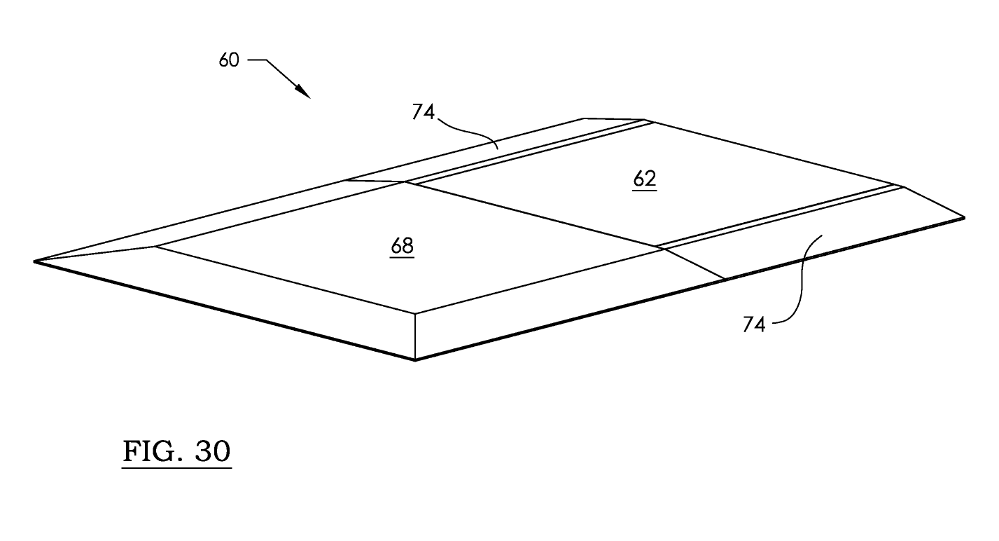

It should be noted that the drawings herein are not to scale.

### DETAILED DESCRIPTION

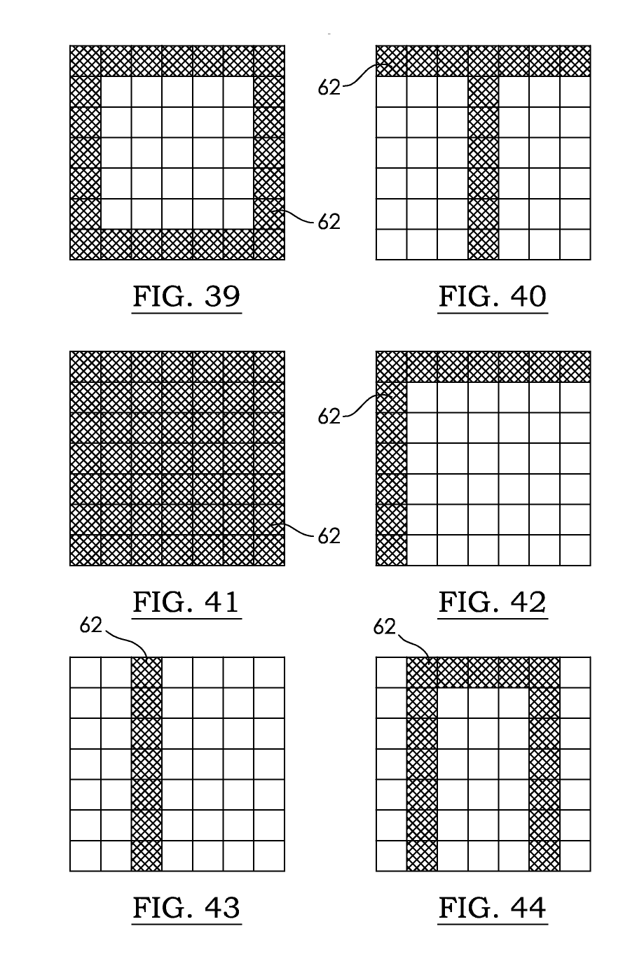

Describing now in further detail these exemplary embodiments with reference to the Figures as described above, a modular instrumented floor covering assembly 60 is used in connection with a subject (not shown) walking across the assembly. The floor covering assembly 60 comprises a plurality of sensor panels 62 having interlocking edges 64. The sensor panels 62 are adapted for interlocking the adjacent panels together along the edges 64. Each sensor panel 62 has a pressure sensor matrix 66 responsive to a weight of the subject for generating data relating to movement of the subject. The plurality of sensor panels 62 are adapted for selective and releasable assembly in patterns, as shown in FIGS. 39-44. The patterns shown can be assembled in combinations as needed. Thus, any pattern of connected square elements can be created. The panels must be laid out in uniform orientation, not rotated with respect to one another. Communicating means is provided for communicating data between adjacent sensor panels and from the sensor panels to an outside computer (not shown).

At least one inert panel 68 is provided, having one interlocking edge 70. The inert panel 68 is adapted for interlocking with one of the sensor panels 62 along the interlocking edge 70. The inert panel 68 has a beveled edge 72 along remaining edges so as to preclude tripping the subject. The inert panel 68 is for guiding the subject toward the sensor panels 62. The subject will take one or two steps on the inert panel 68 before stepping onto the sensor panels 62, to ensure a uniform gait.

At least one edge panel 74 is provided, having one interlocking edge 76. Typically, two edge panels 74, one on each side, will accompany each sensor panel 62 along the entire pattern. This will give the system a finished, non-trip edge. Furthermore, power can be connected to an edge panel 74 anywhere along the entire pattern, as will be explained hereinbelow. The edge panel 74 is adapted for interlocking with one of the sensor panels 62 along the interlocking edge 76. The edge panel 74 has a beveled edge 78 opposing the interlocking edge 76 so as to preclude tripping the subject.

The interlocking edges 64, 70, and 76 each include a channel 80 extending along at least one edge, and in particular, along two edges 64 of each sensor panel 62. The channel, typically an elongated channel strip 80, also extends along one edge 70 of the inert panel 68, and along one edge 76 of the edge panel 74. The channel strip 80 has a U-shaped cross-section with a tapered opening 82 and at least one inside shoulder 84.

An arrow 86 extends along at least one edge, and in particular, along two edges 64 of each sensor panel 62. The arrow, typically an elongated arrow strip 86, also extends along one edge 70 of the inert panel 68, and along one edge 76 of the edge panel 74. The arrow strip 86 has an arrowhead-shaped cross-section with a tapered outer portion 88 and at least one outside shoulder 90. The arrow strip 86 of each panel is releasably inserted into the channel strip 80 of the adjacent panel for interlocking the adjacent panels together. The arrow strip outside shoulder 90 releasably engages the channel strip inside shoulder 84 so as to resist disengaging, as shown in FIGS. 31-34.

The channel strip 80 has an assembly direction 92 defined as facing the opposed arrow strip 86. The arrow strip 86 has an assembly direction 94 defined as facing the opposed channel strip 80.

Each sensor panel 62 defines a polygon having four edges. In the preferred embodiment, each sensor panel 62 defines a square. The elongated channel strip 80 extends along two edges of each sensor panel 62. The elongated arrow strip 86 extends along the remaining two edges of each sensor panel 62.

In the preferred embodiment, the elongated channel strip 80 extends along two adjacent edges of each sensor panel 62. The elongated arrow strip 86 extends along the remaining two adjacent edges of each sensor panel 62.

Each sensor panel 62 includes a generally planar bottom surface 96 and an opposed top surface 98 generally parallel to the bottom surface 96. The channel strip 80 faces away from either the bottom surface or the top surface. In the preferred embodiment, the channel strip 80 extends downward in the assembly direction 92, away from the frame layer 106, and generally perpendicular to the top surface 98.The arrow strip 86 faces away from the opposed one of either the bottom surface 96 or the top surface. In the preferred embodiment, the arrow strip 86 extends upward in the assembly direction 94, away from the base layer 100, and generally perpendicular to the bottom surface 96. The panels are assembled by pressing each panel downward in a generally vertical direction. Handholes 99 are provided in the bottom surface 96.

Each sensor panel 62 includes a generally rigid base layer 100 extending upward from the bottom surface 96. A circuit layer 102 extends upward from the base layer 100. A sensor matrix layer 104 extends upward from the circuit layer 102.

A frame layer 106 extends upward from the sensor matrix layer 104. The frame layer 106 extends perimetrically around the sensor panel 62. The frame layer 106 has an interior space 108. A fill layer 110 extends upward from the sensor matrix layer 104 coextensive with the frame layer 106. The fill layer 110 is composed of flexible material, and is disposed within the frame layer interior space 108.

A cover layer 112 extends upward from the frame layer 106 to the top surface 98. The cover layer 112 is composed of flexible material, and extends across the fill layer 110 and the frame layer 106.

The cover layer 112 and the fill layer 110 will convey the weight of the subject to the sensor matrix layer 104. The rigid or semi-rigid circuit layer 102 and base layer 100 will support the weight of the subject.

At least one circuit board 114 is immersed in the circuit layer 102. The circuit board 114 is operatively electrically connected to the sensor matrix 66 for collecting data from the sensor matrix 66.

At least one transmitter 116 is immersed in the circuit layer 102 and operatively electrically connected to the circuit board 114 for transmitting data wirelessly. Data is transmitted between individual sensor panels 62. Data is also transmitted to an outside computer (not shown) for analysis. Individual sensor panels 62 can be repositioned easily into different patterns due to the wireless communication,

Power means is provided for supplying power to the sensor panels 62 and between adjacent sensor panels 62. The power means comprises at least one pair, and preferably two pairs, of electrical connectors 118 disposed on each edge 64 of each of the sensor panels 62. One of the pair is for positive voltage, and the remaining one of the pair is for negative voltage. The connectors 118 on adjacent sensor panels 62 are operatively electrically and releasably connected together upon interlocking adjacent panels together along the edges.

At least one pair of the electrical connectors 118 is disposed on the interlocking edge 76 of the edge panel. The electrical connectors 118 are adapted for operatively electrically and releasably connecting to the electrical connectors 118 on adjacent sensor panels 62.

A power supply 122 is provided, which operatively electrically and releasably connects to the electrical connectors 118 on the edge panel 74. Additional power supplies 122 can be connected to edge panels 74 wherever convenient, and as needed. For example, a short pattern may need only one power supply 122. A more lengthy pattern requires more power, and hence, a second or third power supply 122 can be connected anywhere along the pattern.

A conductor 120 on the panel interlocking edge 64, 70, and 76 is adapted for contacting a conductor 120 on the adjacent panel interlocking edge with spring bias. Two conductors 120 comprise one electrical connector 118. Wires 124 connect the power supply 122 to the electrical connectors 118. A plug 126 connects the power supply 122 to an electrical source (not shown).

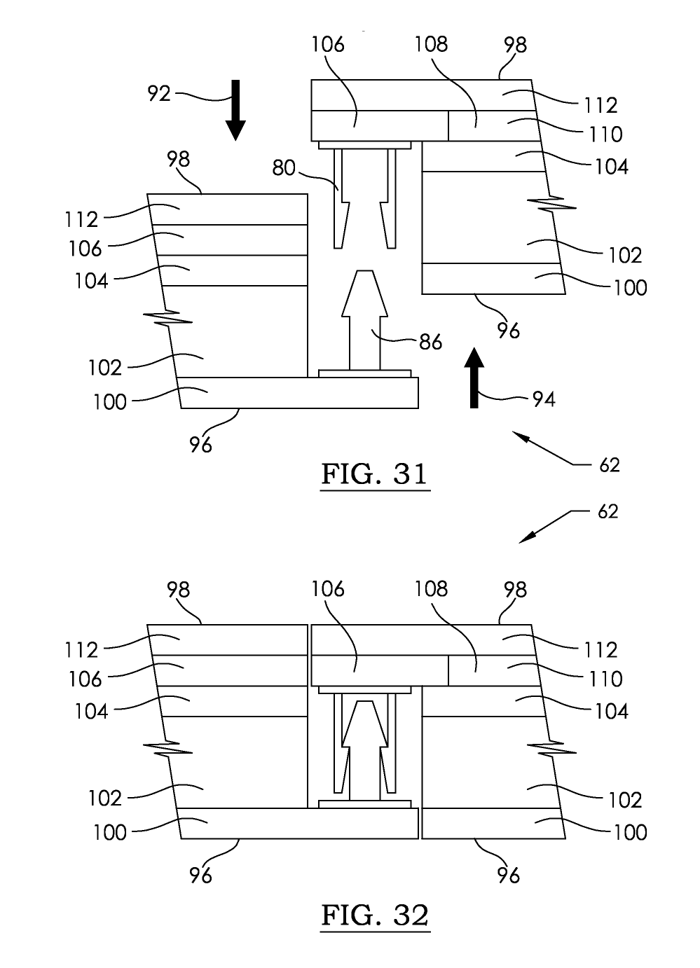

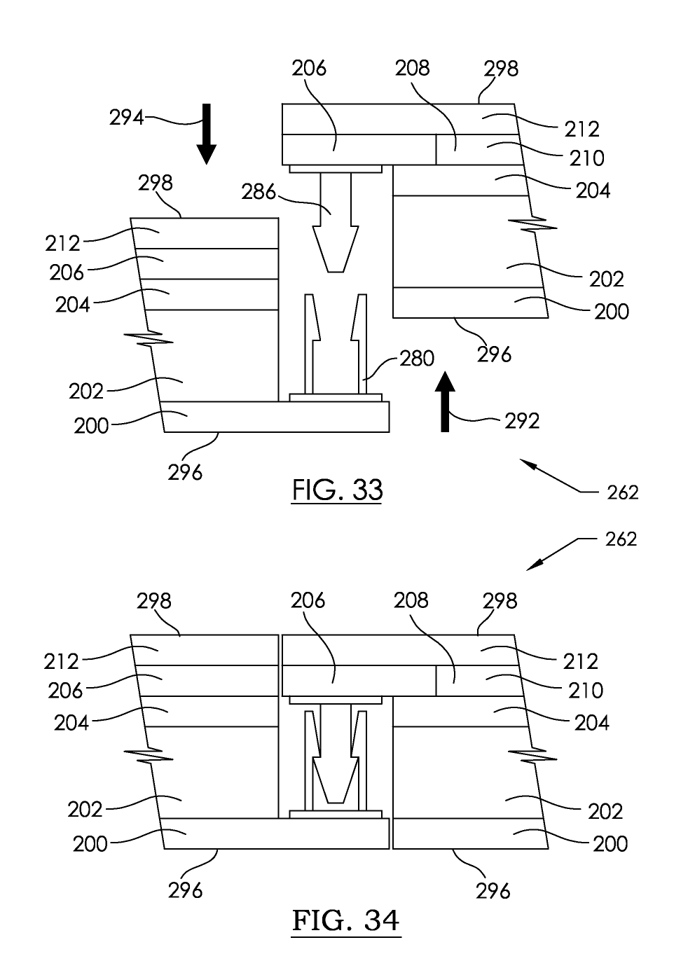

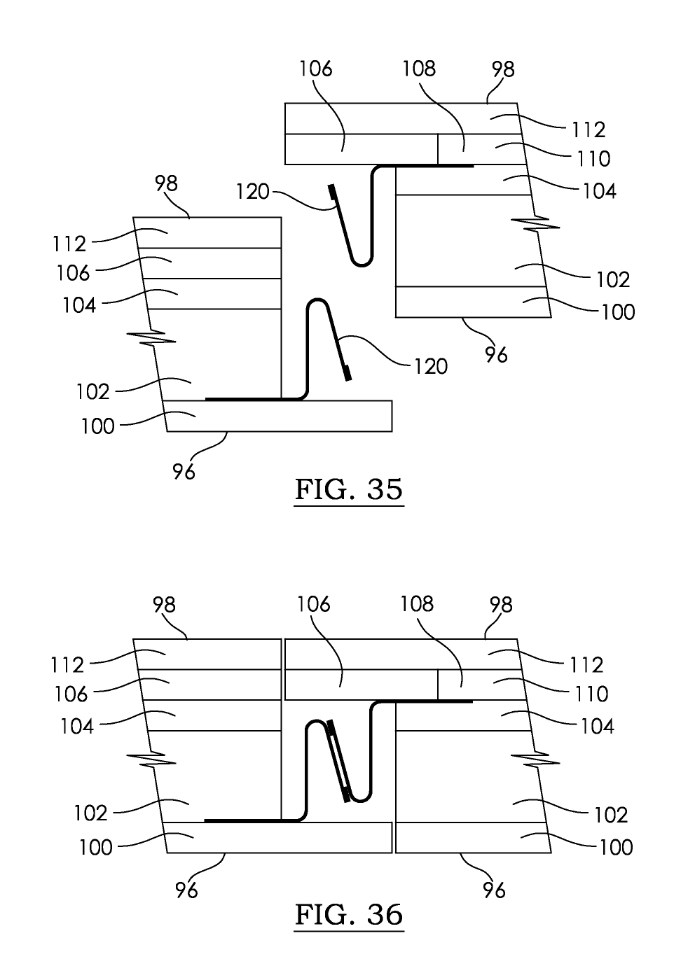

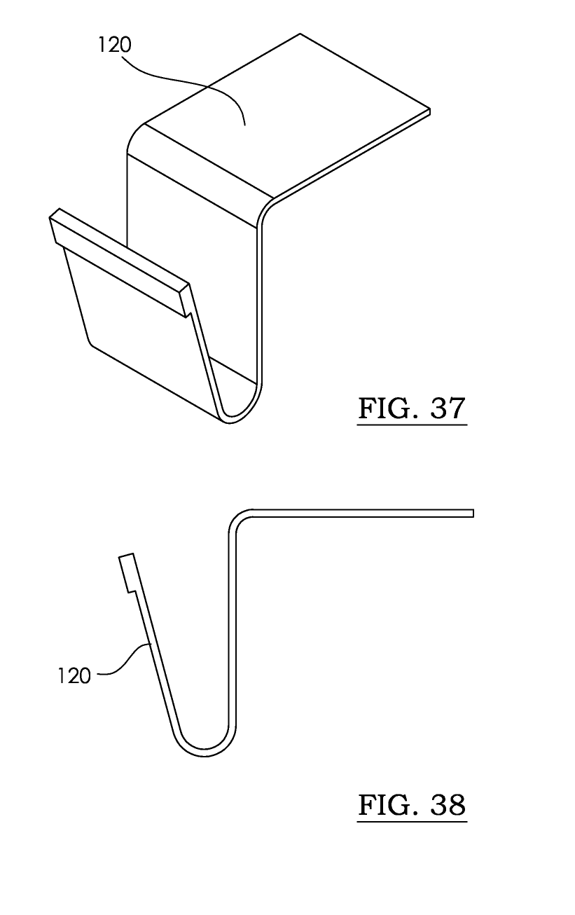

Turning now to FIGS. 33 and 34, in another embodiment constructed in accordance with the invention, each sensor panel 262 is similar to sensor panel 62 described above. Sensor panel 262 includes a generally planar bottom surface 296 and an opposed top surface 298 generally parallel to the bottom surface 296. The channel strip 280 faces away from the bottom surface. This embodiment differs from sensor panel 62 described above, in that the channel strip 280 extends outward in the assembly direction 292, away from the base layer 200, and generally perpendicular to the bottom surface 296.

The arrow strip 286 faces away from the opposed top surface 298. The arrow strip 286 extends outward in the assembly direction 294 generally perpendicular to the top surface 98. The panels are assembled by pressing each panel downward in a generally vertical direction.

Each sensor panel 262 includes a generally rigid base layer 200 extending upward from the bottom surface 296. A circuit layer 202 extends upward from the base layer 200. A sensor matrix layer 204 extends upward from the circuit layer 202.

A frame layer 206 extends upward from the sensor matrix layer 204. The frame layer 206 extends perimetrically around the sensor panel 262. The frame layer 206 has an interior space 208. A fill layer 210 extends upward from the sensor matrix layer 204 coextensive with the frame layer 206. The fill layer 210 is composed of flexible material, and is disposed within the frame layer interior space 208.

A cover layer 212 extends upward from the frame layer 206 to the top surface 298. The cover layer 212 is composed of flexible material, and extends across the fill layer 210 and the frame layer 206.

The cover layer 212 and the fill layer 210 will convey the weight of the subject to the sensor matrix layer 204. The rigid or semi-rigid circuit layer 202 and base layer 200 will support the weight of the subject.

All other aspects of sensor panel 262 are similar to sensor panel 62 described above. The conductors 120, the electrical connections, the communication, and the assembly procedure are similar to that of sensor panel 62. Only the channel strip 280 and the arrow strip 286 are reversed.

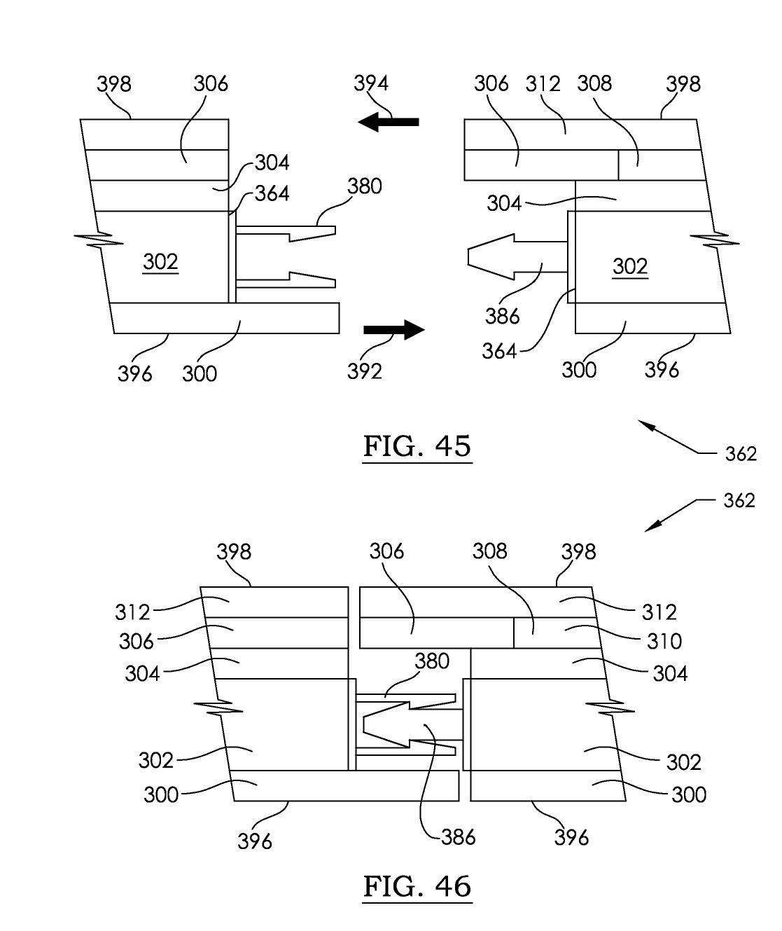

Referring now to FIGS. 45 and 46, in yet another embodiment constructed in accordance with the invention, each sensor panel 362 is similar to sensor panel 62 described above. Sensor panel 362 includes a generally planar bottom surface 396 and an opposed top surface 398 generally parallel to the bottom surface 396. This embodiment differs from sensor panel 62 described above, in that the channel strip 380 extends from the edges 364 outward in the assembly direction 392 generally parallel to the bottom surface 396, as shown in FIGS. 45 and 46.

The arrow strip 386 extends from the edges 364 outward in the assembly direction 394 generally parallel to the bottom surface 396. Thus, the panels 362 are adapted for assembly by pressing each panel sideways in a generally horizontal direction.

Each sensor panel 362 includes a generally rigid base layer 300 extending upward from the bottom surface 396. A circuit layer 302 extends upward from the base layer 300. A sensor matrix layer 304 extends upward from the circuit layer 302.

A frame layer 306 extends upward from the sensor matrix layer 304. The frame layer 306 extends perimetrically around the sensor panel 362. The frame layer 306 has an interior space 308. A fill layer 310 extends upward from the sensor matrix layer 304 coextensive with the frame layer 306. The fill layer 310 is composed of flexible material, and is disposed within the frame layer interior space 308.

A cover layer 312 extends upward from the frame layer 306 to the top surface 398. The cover layer 312 is composed of flexible material, and extends across the fill layer 310 and the frame layer 306.

The cover layer 312 and the fill layer 310 will convey the weight of the subject to the sensor matrix layer 304. The rigid or semi-rigid circuit layer 302 and base layer 300 will support the weight of the subject.

All other aspects of sensor panel 362 are similar to sensor panel 62 described above. The conductors 120, the electrical connections, the communication, and the assembly procedure are similar to that of sensor panel 62. Only the channel strip 380 and the arrow strip 386 are rotated into a horizontal position.

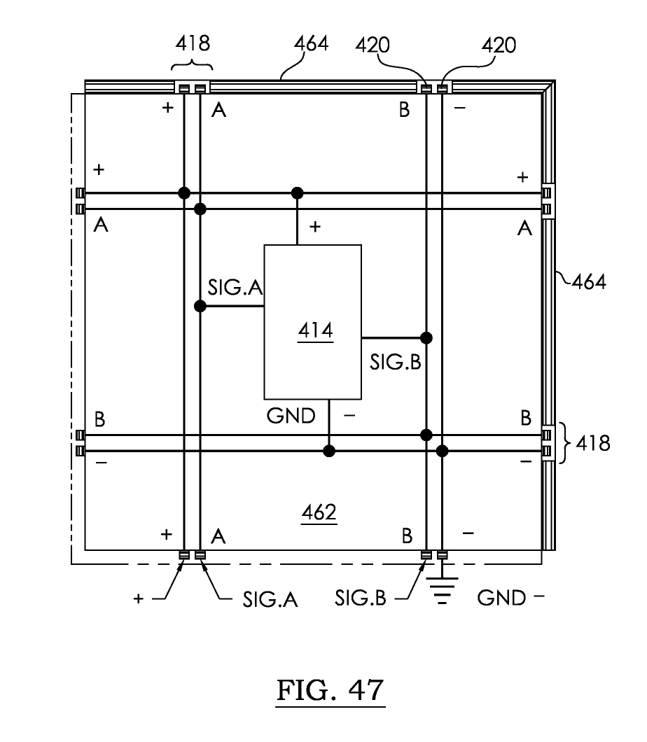

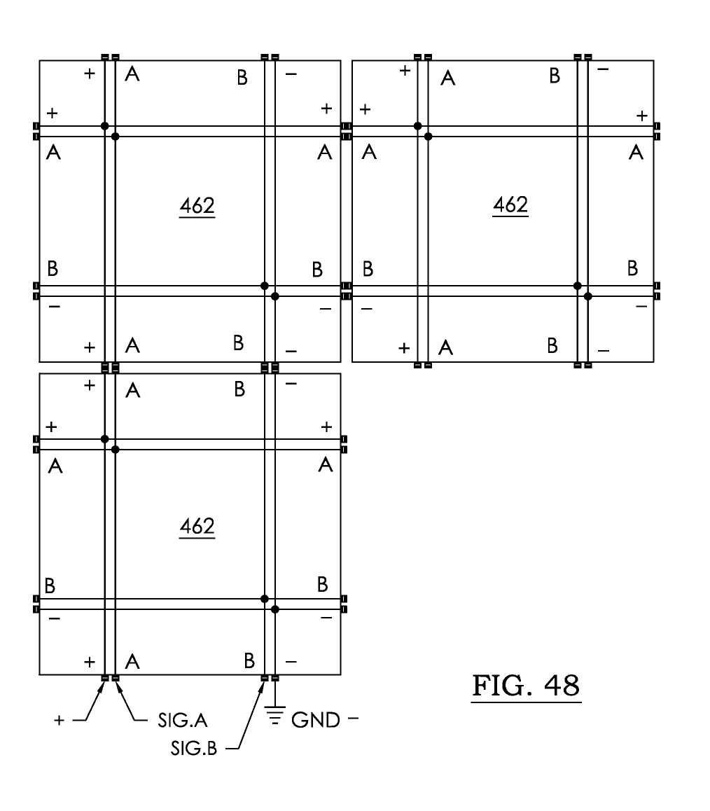

Referring now to FIGS. 47 and 48, in still another embodiment constructed in accordance with the invention, each sensor panel 462 is similar to sensor panel 62 described above. Sensor panel 462 differs from sensor panel 62 in that sensor panel 462 utilizes a “Signal Over Power” method for the transmission of the data or signal, Each pair of electrical connectors 418 has two conductors 420 as before, but they are connected differently. One outside conductor 420 is for positive voltage. The opposite outside conductor 420 is for ground and negative voltage. The two inside conductors 420 are for data. In FIG. 47, signal A and signal B are for data, with the ground being common for both data and power.

FIG. 48 shows how the individual sensor panels 462 are connected together to convey power and signal from each panel to adjacent panels. One example of the protocol that can be used is RS-485, well known to those skilled in the art, All other aspects of sensor panel 462 are similar to sensor panel 62 described above. The conductors 420, arrow strips 86, channel strips 80, and the assembly procedure are similar to that of sensor panel 62. It is to be understood that alternative elements, such as arrow strips 286 and 386, and channel strips 280 and 380, as well as other alternative elements described above, can be utilized with sensor panels 462 and all other sensor panels disclosed, and are to be considered equivalent embodiments within the spirit and scope of the claims.

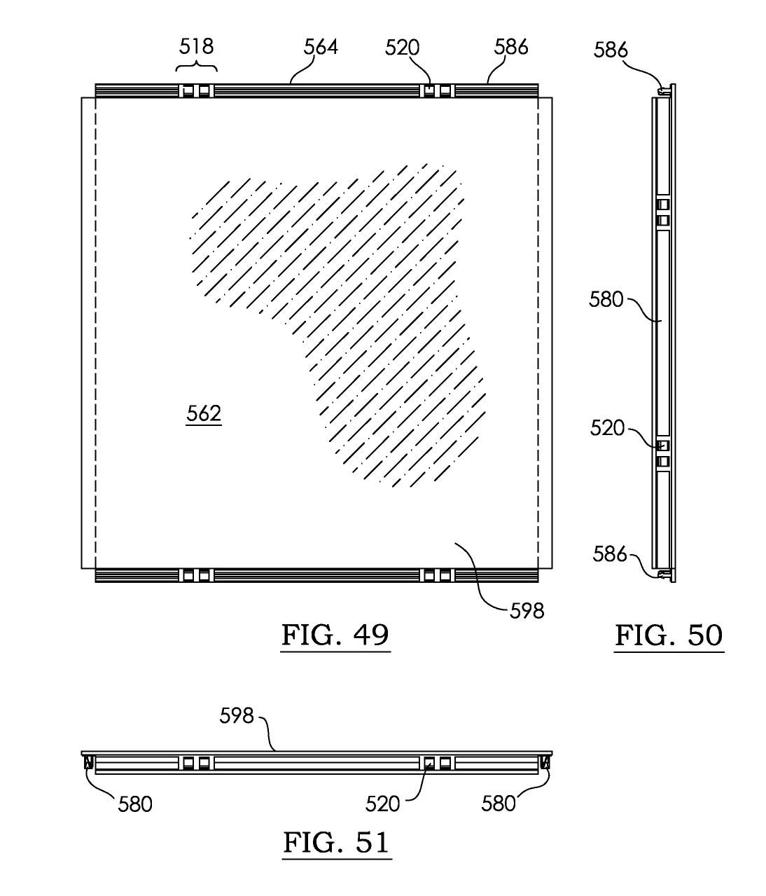

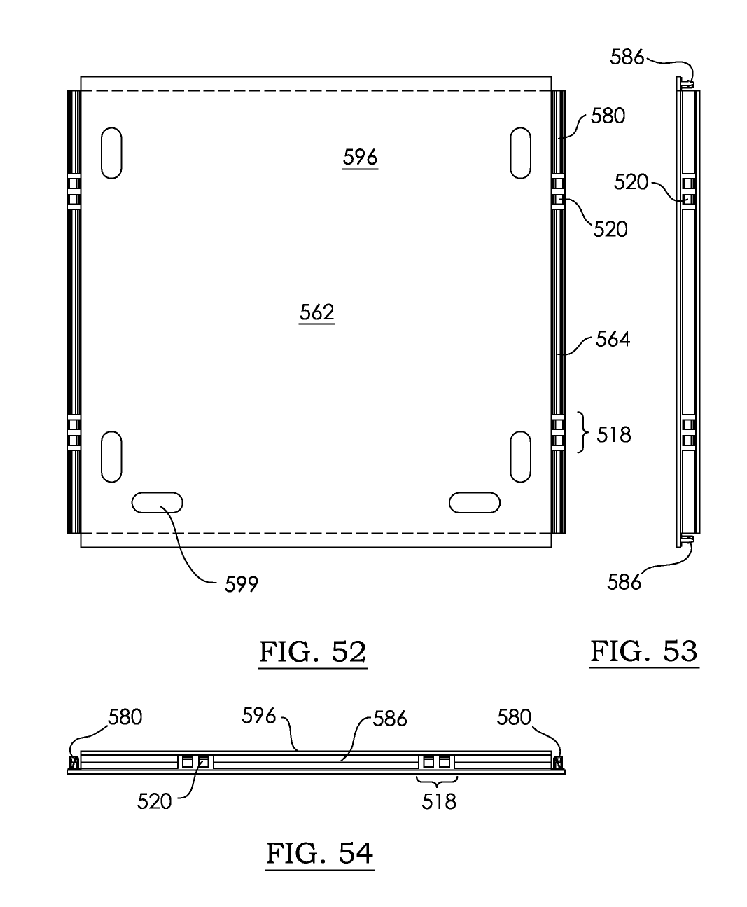

Referring now to FIGS. 49-54, in still another embodiment constructed in accordance with the invention, each sensor panel 562 is similar to sensor panel 62 described above. Sensor panel 562 differs from sensor panel 62 in that the elongated channel strip 580 extends along two opposed edges 564 of each sensor panel 562. The elongated arrow strip 586 extends along the remaining two opposed edges 564 of each sensor panel 562.

All other aspects of sensor panel 562 are similar to sensor panel 62 described above. The electrical connectors 518, conductors 520, arrow strips 586, and channel strips 580, are similar to that of sensor panel 62. The assembly procedure differs in that adjacent panels must be rotated 90° in either direction, so that the opposed arrow strips 586, and channel strips 580, will engage. Other alternative elements described above can be utilized with sensor panels 562, and are to be considered equivalent embodiments within the spirit and scope of the claims.

It will be appreciated that variants of the above-disclosed and other features and functions, or alternatives thereof, may be desirably combined into many other different systems or applications. Various presently unforeseen or unanticipated alternatives, modifications, variations, or improvements therein may be subsequently made by those skilled in the art which are also intended to be encompassed by the following claims.

1. A modular instrumented floor covering assembly for use in connection with a subject walking across the assembly, the floor covering assembly comprising:
a plurality of sensor panels having interlocking edges, the sensor panels being adapted for interlocking adjacent panels together along the edges, each sensor panel having a pressure sensor matrix responsive to a weight of the subject for generating data relating to movement of the subject, the plurality of sensor panels being adapted for selective and releasable assembly in patterns;
communicating means for communicating data from the sensor panels and between adjacent sensor panels; and
power means for supplying power to the sensor panels and between adjacent sensor panels.

2. The modular instrumented floor covering assembly of claim 1, further comprising at least one inert panel having one interlocking edge, the inert panel being adapted for interlocking with one of the sensor panels along the interlocking edge, the inert panel having a beveled edge along remaining edges so as to preclude tripping the subject, the inert panel being adapted for guiding the subject toward the sensor panels.

3. The modular instrumented floor covering assembly of claim 1, further comprising
at least one edge panel having one interlocking edge, the edge panel being adapted for interlocking with one of the sensor panels along the interlocking edge, the edge panel having a beveled edge opposing the interlocking edge so as to preclude tripping the subject.

4. The modular instrumented floor covering assembly of claim 1, wherein the power means further comprises at least one pair of electrical connectors disposed on each edge of each of the sensor panels, a one of the pair being for positive voltage, and a remaining one of the pair being for negative voltage, the connectors on adjacent sensor panels being adapted for operatively electrically and releasably connecting together upon interlocking adjacent panels together along the edges.

5. The modular instrumented floor covering assembly of claim 4, wherein the power means further comprises:
at least one pair of the electrical connectors disposed on the interlocking edge of the edge panel, the electrical connectors being adapted for operatively electrically and releasably connecting to the electrical connectors on adjacent sensor panels; and
a power supply adapted for operatively electrically and releasably connecting to the electrical connectors on the edge panel.

6. The modular instrumented floor covering assembly of claim 5, wherein the electrical connectors further comprise a conductor on the panel interlocking edge adapted for contacting a conductor on the adjacent panel interlocking edge with spring bias.

7. The modular instrumented floor covering assembly of claim 1, wherein the interlocking edges further comprise:
at least one channel extending along at least one edge of each sensor panel, the channel having a U-shaped cross-section with a tapered opening and at least one inside shoulder; and
at least one arrow extending along at least one edge of each sensor panel, the arrow having an arrowhead-shaped cross-section with a tapered outer portion and at least one outside shoulder, the arrow of each panel being adapted for releasable insertion into the channel of the adjacent panel for interlocking the adjacent panels together, the arrow outside shoulder being adapted to releasably engage the channel inside shoulder so as to resist disengaging; wherein
the channel has a channel assembly direction defined as facing the opposed arrow, and the arrow has an arrow assembly direction defined as facing the opposed channel.

8. The modular instrumented floor covering assembly of claim 7, further comprising:
each sensor panel defines a polygon having four edges;
the channel is an elongated channel strip extending along two edges of each sensor panel; and
the arrow is an elongated arrow strip extending along the remaining two edges of each sensor panel.

9. The modular instrumented floor covering assembly of claim 7, further comprising:
each sensor panel defines a square;
the channel extends along two adjacent edges of each sensor panel; and
the arrow extends along the remaining two adjacent edges of each sensor panel.

10. The modular instrumented floor covering assembly of claim 7, further comprising:
each sensor panel defines a square;
the channel extends along two opposed edges of each sensor panel; and
the arrow extends along the remaining two opposed edges of each sensor panel.

11. The modular instrumented floor covering assembly of claim 8, further comprising:
each sensor panel includes a generally planar bottom surface and an opposed top surface generally parallel to the bottom surface;
the channel strip faces away from one of the bottom surface and the top surface, the channel strip extending outward in the channel assembly direction generally perpendicular to one of the bottom surface and the top surface respectively; and
the arrow strip faces away from the opposed one of the bottom surface and the top surface, the arrow strip extending outward in the arrow assembly direction generally perpendicular to one of the bottom surface and the top surface respectively; so that
the panels are adapted for assembly by pressing each panel downward toward the opposed panel in a generally vertical direction.

12. The modular instrumented floor covering assembly of claim 8, further comprising:
each sensor panel includes a generally planar bottom surface and an opposed top surface generally parallel to the bottom surface;
the channel strip extends from the edges outward in the channel assembly direction generally parallel to the bottom surface; and
the arrow strip extends from the edges outward in the arrow assembly direction generally parallel to the bottom surface; so that
the panels are adapted for assembly by pressing each panel sideways toward the opposed panel in a generally horizontal direction.

13. The modular instrumented floor covering assembly of claim 1, wherein each sensor panel further comprises:
a generally planar bottom surface and an opposed top surface generally parallel to the bottom surface;
a generally rigid base layer extending upward from the bottom surface;
a circuit layer extending upward from the base layer;
a sensor matrix layer extending upward from the circuit layer;
a frame layer extending upward from the sensor matrix layer, the frame layer extending perimetrically around the sensor panel, the frame layer having an interior space;
a fill layer extending upward from the sensor matrix layer coextensive with the frame layer, the fill layer being disposed in the frame layer interior space, the fill layer being flexible material; and
a cover layer extending upward from the frame layer to the top surface, the cover layer extending across the fill layer and the frame layer, the cover layer being flexible material; so that
the cover layer and the fill layer will convey the weight of the subject to the sensor matrix layer, and the circuit layer and the base layer will support the weight of the subject.

14. The modular instrumented floor covering assembly of claim 13, wherein each sensor panel further comprises:
at least one circuit board immersed in the circuit layer and operatively electrically connected to the sensor matrix for collecting data from the sensor matrix; and
at least one transmitter immersed in the circuit layer and operatively electrically connected to the circuit board for transmitting data wirelessly.

15. The modular instrumented floor covering assembly of claim 14, wherein the selective and releasable assembly in patterns is further defined by software.

16. The modular instrumented floor covering assembly of claim 14, wherein the selective and releasable assembly in patterns further comprises patterns and enjoined combinations of patterns selected from the group consisting of:
a straight pattern;
a T-shaped pattern;
an L-shaped pattern;
a U-shaped pattern;
an area pattern; and
a perimeter pattern.

17. The modular instrumented floor covering assembly of claim 4, wherein the electrical connectors further comprise a conductor on the panel interlocking edge adapted for contacting a conductor on the adjacent panel interlocking edge with spring bias.

18. A modular instrumented floor covering assembly for use in connection with a subject walking across the assembly, the floor covering assembly comprising:
a plurality of sensor panels having interlocking edges, the sensor panels being adapted for interlocking adjacent panels together along the edges, each sensor panel having a pressure sensor matrix responsive to a weight of the subject for generating data relating to movement of the subject, the plurality of sensor panels being adapted for selective and releasable assembly in patterns;
at least one channel extending along at least one edge of each sensor panel, the channel having a U-shaped cross-section with a tapered opening and at least one inside shoulder, the channel defining a one of the interlocking edges;
at least one arrow extending along at least one edge of each sensor panel, the arrow having an arrowhead-shaped cross-section with a tapered outer portion and at least one outside shoulder, the arrow defining another one of the interlocking edges, the arrow of each panel being adapted for releasable insertion into the channel of the adjacent panel for interlocking the adjacent panels together, the arrow outside shoulder being adapted to releasably engage the channel inside shoulder so as to resist disengaging;
at least one pair of electrical connectors disposed on each edge of each of the sensor panels, a one of the pair being for positive voltage, and a remaining one of the pair being for negative voltage, the connectors on adjacent sensor panels being adapted for operatively electrically and releasably connecting together upon interlocking adjacent panels together along the edges, each one of the pair of electrical connectors having a conductor on the panel interlocking edge adapted for contacting a conductor on the adjacent panel interlocking edge with spring bias, the electrical connectors being adapted for supplying power to the sensor panels and between adjacent sensor panels; and
communicating means for wirelessly communicating data between adjacent sensor panels and from the sensor panels to a computer for analysis.
